(19) United States
(12) Patent Application Publication
(10) Pub. No.: US 2009/0221013 A1
(43) Pub. Date: Sep. 3, 2009

(54) METHOD FOR QUANTIFYING LIVING COLIFORM MICROORGANISMS IN A WATER SAMPLE

(75) Inventors: Anne-Sophie Lepeuple, Andresy (FR); Philipe Lebaron, Banyuls Sur Mer (FR); Pierre Servais, Charleroi (BE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Veolia Eau-Compagnie Generale Des Eaux, Paris (FR)

(21) Appl. No.: 11/916,326

(22) PCT Filed: Jun. 1, 2006
(86) PCT No.: PCT/FR2006/050497

§ 371 Date: Sep. 19, 2008

(30) Foreign Application Priority Data
Jun. 3, 2005 (FR) 0505618

(51) Int. Cl. C12Q 1/34

(52) U.S. Cl. 435/18

(57) ABSTRACT

The invention relates to a method of rapidly quantifying living coliform microorganisms in a sample of water in which the microorganisms are put into contact with a fluorogenic substrate that is hydrolyzed in methylumbelliferone by an enzyme given off by the microorganisms. Methylumbelliferone is detected by the fluorescence that it emits, with the rate at which this fluorescence varies being measured in order to determine the concentration of coliform microorganisms in the original sample on the basis of the calculated rate of variation and a correlation plot. In characteristic manner, a preliminary treatment step is performed on the sample, in particular pre-filtering, serving to remove at least some of the particles in suspension in the water, thus making it possible to achieve quantification that is entirely comparable with the quantification given by the standardized multi-well panel method. The invention is applicable to analyzing bathing water for detecting Escherichia coli.

The invention relates to a method of rapidly quantifying living coliform microorganisms in a sample of water, and to the use of the method.

In particular, but in non-limiting manner, the present invention relates to the method of rapidly quantifying Escherichia coli microorganisms in a sample of bathing water, and in particular of sea water.

In the context of monitoring bathing water quality, the analyses usually performed relate essentially to microbiological parameters and mainly to searching for indicator bacteria that are representative of fecal pollution (fecal coliforms such as Escherichia coli and enterococci), regardless of whether its origin is human or animal (warm-blood animals). The presence of such germs in water indicates that it has been contaminated by fecal matter and implies that pathogenic germs are potentially present. Thus, regulations concerning bathing water quality set guide values and mandatory values corresponding to two concentration ceilings for these indicators in the waters that have been analyzed, and serving to evaluate the microbiological quality of such waters.

At present, the standardized methods used for enumerating fecal coliforms and enterococci rely for the most part on Pasteur's methods. Some methods are based on culturing microorganisms on selected culture media and then quantifying them after an incubation period lying in the range 24 hours to 48 hours. Other methods are based on enzymatic methods that rely on detecting an enzyme or a combination of enzymes under selective growth conditions. Measurements are performed on multi-well panels and the results rely on a statistical estimate of the concentration in target germs. These tests are presently in routine use, and they represent the reference method in the field of analyzing bathing waters.

One of the stakes in the field of microbiological diagnosis, and in the field of analyzing bathing water in particular, is to develop methods that are alternative to culturing methods and that are faster.

Patent EP 0 386 051 proposes a method of quantifying total coliform microorganisms by measuring specific enzymatic activity (β-D-galactosidase activity) under standardized conditions of pH, temperature, and substrate concentration.

The following reaction takes place: hydrolysis of 4-methylumbelliferyl-β-D-galactoside (MuGala) in β-D-galactosic acid and in (fluorescent) 4-methylumbelliferone (MUF) catalyzed by the β-D-galactosidase enzyme. The rate at which the products of that reaction are formed is proportional, under standardized conditions, to the quantity of enzyme that is naturally present in the reaction medium. Working on the principle that the quantity of enzyme is constant within bacteria of the species forming the total coliform microorganisms, measuring enzymatic activity (monitoring the rate at which (fluorescent) 4-methylumbelliferone is formed by spectrofluorimetry) makes it possible to determine the total number of coliform microorganisms in a sample of water, and thus to deduce their concentration.

In particular, EP 0 386 051 provides for adding ingredients suitable for increasing enzymatic activity in order to activate fluorescence and make it easier to measure the rate at which fluorescence varies under circumstances in which the concentration of microorganisms is low, without waiting for the microorganisms to multiply by reproduction over several generations.

It is also known (“Use of enzymatic methods for rapid enumeration of coliforms in fresh waters”, Journal of Applied Microbiology 2000, 88, 404-413, George et al.) to perform rapid quantification of living coliform microorganisms constituted by Escherichia coli in a sample of water.

Under such circumstances, the fluorogenic substrate used is 4-methylumbelliferyl-β-D-glucuronide (MuGlu), which is catalyzed by the β-D-glucuronidase enzyme in β-D-glucuronic acid and in 4-methylumbelliferone (MUF) of which the fluorescence is measured.

There also exists a standardized method of enumerating Escherichia coli in water (ISO 9308-3) also referred to the “multi-well panel” method, which acts as a reference in regulations relating to the microbiological quality of surface waters, in particular bathing waters, for determining contamination levels.

In that standardized ISO 9308-3 method, the sample diluted in a plurality of dilutions is seeded into a series of wells in a panel containing the dehydrated culture medium and the substrate for the enzyme 4-methylumbelliferyl-β-D-glucuronide (MuGlu). After an incubation period lying in the range 36 hours (h) to 72 h at 44° C., the wells of the panel are examined under ultraviolet radiation at a wavelength of 366 nanometers (nm). The presence of Escherichia coli in a given well is indicated by blue fluorescence that results from the hydrolysis of MuGlu. For each dilution, the number of positive wells is noted. The results are given in terms of the most probable number (MPN) per 100 milliliters (mL) (where MPN is a statistical estimate of microorganism density, which is assumed to satisfy a Poisson distribution in the seeded volumes).

It should be observed that that standardized method is particularly well adapted to waters that are rich in material in suspension.

Nevertheless, that standardized method using a multi-well panel requires on average 48 h, whereas the rapid methods of detecting Escherichia coli as described in the prior art enable a result to be obtained in 1 h and can be used to be give a rapid indication concerning the microbiological quantity of the water, and thus as a tool for handling immediate health risks.

Nevertheless, the methods for detecting Escherichia coli rapidly do not always give results that are compatible with the standardized method using a multi-well panel. In particular, with water that is charged with material in suspension such as sand or sediments, rapid methods give a result that is higher than that of the standardized method.

An object of the present invention is to provide a method of rapidly quantifying living coliform microorganisms in a sample of water, that enables the drawbacks of the prior art to be overcome, in particular by making it possible to obtain results that are comparable with those of the standardized method, with any type of water, and in particular with turbid water that rich in materials in suspension.

Below in the present specification and in the meaning of the present invention, the term “rapid” means a method that can be carried out in a few hours at most, and in particular in 1 h at most.

To this end, the present invention provides a method that is characterized in that it comprises the following steps:

a) performing a preliminary step of sample treatment enabling at least some of the particles in suspension in the water to be removed;

b) filtering the sample on a filter having pores of a size smaller than the size of coliform microorganisms, so as to concentrate the coliform microorganisms on the filter;

c) placing the filter retaining the coliform microorganisms in a receptacle containing a buffer solution;

d) raising the receptacle to an activity temperature for the measured enzyme;

e) adding a fluorogenic substrate to the receptacle that is suitable for forming 4-methylumbelliferone (MUF) by hydrolysis under the effect of the enzyme;

f) at regular intervals measuring the quantity of fluorescence emitted by:
f1) taking a test quantity of the reaction medium contained in said receptacle;
f2) adding an agent for modifying pH to increase fluorescence;
f3) exposing the taken test quantity to light having a wavelength close to the excitation wavelength for 4-methylumbelliferone (MUF); and
f4) measuring the fluorescence emitted by the 4-methylumbelliferone (MUF) produced by the fluorogenic substrate under the effect of the enzyme contained in the coliform microorganisms;

g) calculating the rate at which the emitted fluorescence varies; and

h) determining the concentration of coliform microorganisms in the original sample from the calculated rate of variation and from a plot correlating rate of variation as a function of concentration in coliform microorganisms.

In this way, it will be understood that the presence of the preliminary treatment step performed on the sample enables at least some of the particles in suspension in the water to be removed, thereby, where appropriate, reducing the turbidity of the water and thus making it possible with this rapid detection method to obtain a result that is comparable to that obtained by the standardized method using a multi-well panel, regardless of the turbidity of the water to be tested.

In addition to the simplicity with which the preliminary step can be performed, this solution also presents the additional advantage of not requiring a long time for its performance.

Overall, using the solution of the present invention, it is possible in very simple manner to perform a method of rapid quantification of living microorganisms that gives a result that is comparable with the standardized method.

The method of the invention includes one or more of the following preferred dispositions:
the preliminary treatment step comprises pre-filtering;
the pre-filtering is performed using at least one filter presenting pores of a size greater than or equal to 8 micrometers (μm);
the pre-filtering is performed with a first filter and with a second filter that presents a pore size smaller than the first filter, and that is placed upstream from the first filter;
the pre-filtering is performed with a first filter presenting a pore size greater than or equal to 15 μm (advantageously substantially equal to 20 μm) and a second filter presenting a pore size greater than or equal to 8 μm (advantageously substantially equal to 10 μm);
the first filter and the second filter are superposed; and
the filter(s) is/are made of a material belonging to the group comprising: Nylon (registered trademark); polycarbonate; cellulose acetate; sulfone; polyvinylidene fluoride (PVDF); polyethersulfone (PES); and polyacrylonitrile; or more generally any type of synthesized polymer.

Other advantages and characteristics of the invention appear on reading the following description made by way of example and with reference to the accompanying drawings, in which:

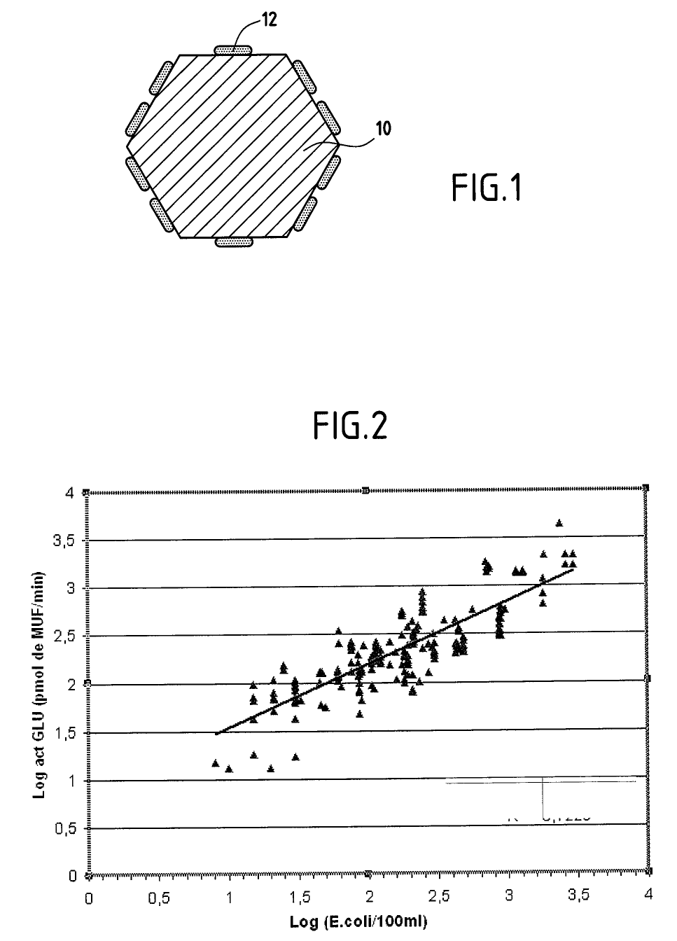

FIG. 1 is a diagram of a particle in suspension in the water of the sample;

FIG. 2 shows the correlation between the rate of variation of fluorescence and the concentration of Escherichia coli bacteria in sea water; and

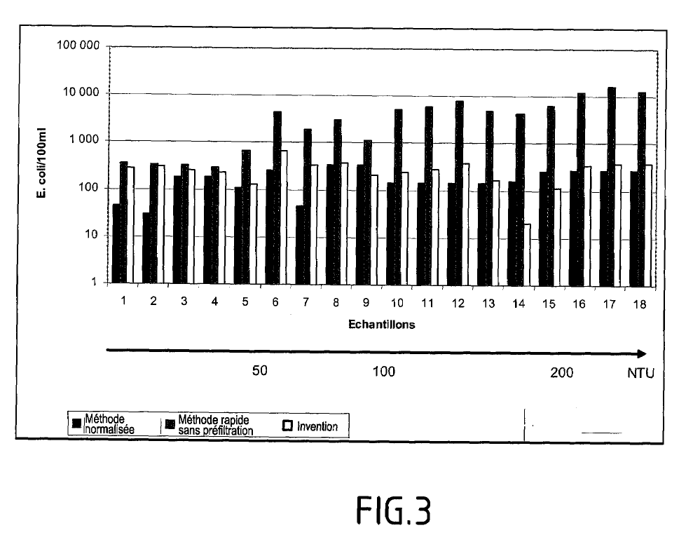

FIG. 3 is a bar chart showing the results obtained using the standardized method, using the rapid method of the prior art, and using the invention.

FIG. 1 is a diagram representing a particle 10, e.g. a grain of sand, that might be in suspension in the water of the sample for which it is desired to measure the concentration in Escherichia coli bacteria.

The particle 10 carries on its surface ten Escherichia coli bacteria 12.

When implementing the standardized quantification method, particles of material in suspension having bacteria cells of the Escherichia coli type adhering thereto are distributed in the well of multi-well panels. Each particle 10 drops into a well, causes that well to be counted as being positive, i.e. statistically containing a single cell of Escherichia coli, whereas in reality the number of Escherichia coli is greater (ten for the particle 10 of FIG. 1).

In contrast, in the rapid enzyme method, it is overall activity that is measured. Thus, all of the Escherichia coli bacteria cells attached to the particles present measurable enzyme activity.

That is why the rapid enzyme method of the prior art gives a higher result than the result given by the standardized quantification method using multi-well panels, given that all of the E. coli cells are enumerated, including each of the cells attached to pieces of material in suspension.

The present invention is thus based on the principle of removing all or some of the particles in suspension from the water in order to quantify living coliform microorganisms, and in particular Escherichia coli, individually, without taking into consideration those that are grouped together on a particle.

As an example of the present invention, tests have been carried out on sea water. However the method of the invention is not restricted to being used with sea water only, but can be used equally well to quantify living coliform microorganisms in fresh water.

In this example, the enzyme is β-D-glucuronidase, the fluorogenic substrate is 4-methylumbelliferyl-β-D-glucuronide (MuGlu) in order to quantify the living coliform Escherichia coli microorganisms, and the pH modifier agent is sodium hydroxide in order to obtain a pH greater than 10.

Furthermore, in this example, the preliminary treatment step that is used on the sample for removing at least some of the particles in suspension in the water is pre-filtration.

To this end, the sample of water for analysis is filtered through two superposed filters. The first situated on top is made of Nylon (registered trademark) with pore size of 20 μm. The second filter situated underneath is made of polycarbonate and has pore size of 10 μm.

It is the filtrate coming from the pre-filtration step that is used to perform rapid measurement of enzymatic activity.

In this example, the objective is to measure the quantity of bacteria of the Escherichia coli type.

A filtration step is then performed in order to concentrate the Escherichia coli on a filter.

This filtration step consists in passing 100 mL of sample of the filtrate onto a polycarbonate filter having a diameter of 47 millimeters (mm) and pore size of 0.22 μm.

Before the end of filtering, the walls of the funnel are rinsed in distilled water (or pyrogen-free water), and then the polycarbonate filter is placed in a receptacle containing a phosphate buffer (pH 6.9) at 44° C. (thermostatically-controlled water-bath).

Thereafter, enzymatic activity proper is measured.

To do this, the solution of 4-methylumbelliferyl-β-D-glucuronide (MuGlu 1.1 milligrams per milliliter (mg/mL), Triton X100 0.04% vol/vol) is added to the receptacle containing the reaction mixture.

After about 1 minute (min), the fluorescence emitted by the reaction mixture is measured over time (by taking a sample every 5 min over about 30 min), the pH of the mixture in the sample being previously adjusted to 10.6 by adding sodium hydroxide (NaOH).

Light is used having an excitation wavelength of 362 nm and an emission wavelength of 445 nm.

Finally, the data is processed, i.e. the fluorescence measurements.

That consists in calculating the slope of the line representing variation in the concentration of MUF in nano-moles per liter (nmol/L) as a function of time, which is representative of enzymatic activity (pmol of MUF/minute).

The concentration in E. coli (number of E. coli per 100 mL) is determined using the correlation line for enzymatic activity against multi-well panel enumeration. The number of E. coli in the 100 mL of sample is calculated at the same time as the enzymatic activity.

This correlation line is plotted in FIG. 2 for sea water: it is the result of logarithmic linear regression between GLUase activity and Escherichia coli concentration for sea water samples from a site in the Mediterranean (Pyrénées-Orientales, France), i.e. Log(GLUase act. pmoles/min. 100 mL)=0.6534 Log (Escherichia coli/100 mL)+0.8.856 (r²=0.7229).

FIG. 3 shows the results obtained using all three methods: the standardized method, the rapid enzymatic method without pre-filtering, and the method of the invention, i.e. the rapid enzymatic method with the pre-filtering step performed on samples of sea water having turbidity lying in the range 34 formazin nephelometric units (FNU) to 223 FNU.

The results show that the greater the turbidity, the more the pre-filtering step is crucial for obtaining results close to those obtained using the standardized method so as to provide an indication of microbiological quality complying with regulations.

On the basis of these results, it can be understood that the method of the invention can be implemented regardless of the turbidity of the water, and that it is very advantageous for analyzing water presenting turbidity greater than or equal to 35 FNU, advantageously greater than 50 FNU.

In the preferred example above, two filters were used (respectively made of Nylon (registered trademark) with pore size of 20 μm, and then of polycarbonate with pore size of 10 μm), but other tests have shown that results can be obtained when pre-filtering is performed with a single filter, e.g. a filter having pore size of 8 μm, and in particular a filter made of polycarbonate.

It should be observed that the second polycarbonate filter with pore size of 10 μm could be replaced by a filter having pore size of 8 μm.

The use of two superposed filters in a common pre-filtration unit is equivalent to performing two pre-filtrations in succession.

The filter(s) selected for the pre-filtering step depends on the material and on the size of the pores (cut-off threshold), it being understood that it is necessary to avoid the cut-off threshold being too small since that would lead to clogging or could even retain bacteria constituting living coliform microorganisms to be quantified.

The present invention is not limited to implementing preliminary treatment consisting in performing pre-filtration.

It is possible to remove at least some of the particles in suspension in the water by some other means.

In particular, this preliminary treatment step may include or consist of centrifuging at low speed.

This can serve to separate the particles in suspension from the filtrate.

This preliminary treatment step may also include or consist in using sedimentation or settling, i.e. allowing particles to drop naturally by allowing the sample to stand, and then recovering the supernatent for measurement purposes.

Such a preliminary step of sedimentation may be combined with a physicochemical phenomenon of aggregation (flocculation or coagulation).

It should be observed that the present invention proposes a preliminary treatment step making it possible to match the results obtained by the rapid method of the prior art to the results obtained by the standardized method.

Nevertheless, in order to bring these two measurements closer together, it is possible to envisage adapting the results obtained by the standardized method to the results obtained by the rapid method of the prior art. For this purpose, it is possible for example to separate the bacteria fixed on particles prior to performing analysis using the standardized method, so as to count all of the bacteria. When performing pretreatment by separation, the fraction of the sample measured by both methods is the fraction of non-fixed bacteria and the fraction of bacteria fixed to particles of matter in suspension.

Such separation may be implemented by various physicochemical methods: ultrasound treatment; the use of detergents; . . . .

1. A method of rapid quantification of living coliform microorganisms in a sample of water, the method being characterized in that it comprises the following steps:
a) performing a preliminary step or sample treatment enabling at least some of the particles in suspension in the water to be removed;
b) filtering the sample on a filter having pores of a size smaller than the size of coliform microorganisms, so as to concentrate the coliform microorganisms on the filter;
c) placing the filter retaining the coliform microorganisms in a receptacle containing a buffer solution;
d) raising the receptacle to an activity temperature for the measured enzyme;
e) adding a fluorogenic substrate to the receptacle that is suitable for forming 4-methylumbelliferone (MUF) by hydrolysis under the effect of the enzyme;
f) at regular intervals measuring the quantity of fluorescence emitted by:
f1) taking a test quantity of the reaction medium contained in said receptacle;
f2) adding an agent for modifying pH to increase fluorescence;
f3) exposing the taken test quantity to light having a wavelength close to the excitation wavelength for 4-methylumbelliferone (MUF); and
f4) measuring the fluorescence emitted by the 4-methylumbelliferone (MUF) produced by the fluorogenic substrate under the effect of the enzyme contained in the coliform microorganisms;
g) calculating the rate at which the emitted fluorescence varies; and
h) determining the concentration of coliform microorganisms in the original sample from the calculated rate of variation and from a plot correlating rate of variation as a function of concentration in coliform microorganisms.

2. A method according to claim 1, characterized in that said preliminary treatment step comprises pre-filtering.

3. A method according to claim 2, characterized in that the pre-filtering is performed using at least one filter presenting pores of a size greater than or equal to 8 μm.

4. A method according to claim 2, characterized in that the pre-filtering is performed with a first filter and with a second filter that presents a pore size smaller than the first filter, and that is placed downstream from the first filter.

5. A method according to claim 4, characterized in that the pre-filtering is performed with a first filter presenting a pore size greater than or equal to 15 μm and a second filter presenting a pore size greater than or equal to 8 μm.

6. A method according to claim 5, characterized in that said first filter presents a pore size substantially equal to 20 μm and said second filter presents a pore size substantially equal to 10 μm.

7. A method according to claim 5, characterized in that the first filter and the second filter are superposed.

8. A method according to claim 3, characterized in that said filter is made of a material from the group comprising Nylon (registered trademark); polycarbonate; cellulose acetate; sulfone; polyvinylidene fluoride (PVDF); polyethersulfone (PES); and polyacrylonitrile.

9. A method according to claim 1, characterized in that said preliminary treatment step comprises low speed centrifuging.

10. A method according to claim 1, characterized in that said preliminary treatment step comprises sedimentation and recovering a supernatant.

11. A method according to claim 1, characterized in that said enzyme is β-D-glucuronidase, in that said fluorogenic substrate is 4-methylumbelliferyl-β-D-glucuronide (MuGlu) for quantifying living coliform Escherichia coli microorganisms, and in that said pH modifier agent is sodium hydroxide.

12. The use of the method according to claim 1, for analyzing water presenting turbidity greater than 35 FNU.

13. The use of the method according to claim 1, for analyzing a sample of sea water or of fresh water.
